(19) United States
(12) Patent Application Publication
(10) Pub. No.: US 2024/0385420 A1
(43) Pub. Date: Nov. 21, 2024

(54) LIGHT FLUX CONTROLLING MEMBER, LIGHT EMITTING DEVICE, IRRADIATION DEVICE, STERILIZATION DEVICE

(71) Applicant: Enplas Corporation, Saitama (JP)

(72) Inventor: Masayo TAKIZAWA, Saitama (JP)

(73) Assignee: Enplas Corporation, Saitama (JP)

(21) Appl. No.: 18/663,292

(22) Filed: May 14, 2024

(30) Foreign Application Priority Data
May 16, 2023 (JP) 2023-080870

(51) Int. Cl. G02B 17/08; A61L 2/10; A61L 2/26

(52) U.S. Cl. CPC G02B 17/086; A61L 2/10; A61L 2/26; A61L 2202/11

(57) ABSTRACT

A light flux controlling member includes: an incidence surface, two total reflection surfaces, two first emission surfaces, and a second emission surface. The two total reflection surfaces are each a part of a surface that is obtained through rotation with a virtual half straight line as a rotation axis. The two virtual half straight lines each intersect the first emission surface. In plan view of the light flux controlling member, one ends of the two virtual half straight lines are located at the same point on the second axis, and an angle between the two virtual half straight lines is smaller than 180°. The angle between the two virtual half straight lines is formed on a side on which an area of the second emission surface is larger with respect to the first axis.

This application claims the benefit of priority of Japanese Patent Application No. 2023-080870, filed on May 16, 2023, the disclosure of which including the specification, drawings and abstract is incorporated herein by reference in its entirety.

### TECHNICAL FIELD

The present invention relates to a light flux controlling member, a light emitting device, an irradiation device, and a sterilization device.

### BACKGROUND ART

Some transmission image display devices such as liquid crystal display devices use a direct surface light source device as a backlight. In recent years, direct surface light source devices that include a plurality of light emitting elements as the light source have been increasingly used.

For example, a direct surface light source device includes a substrate, a plurality of light emitting elements, a plurality of light flux controlling members (lenses), and a light diffusion member. The light emitting element is a light-emitting diode (LED) such as a white light-emitting diode, for example. The plurality of light emitting elements is disposed in a matrix on a substrate. A light flux controlling member that spreads light emitted from each light emitting element in the surface direction of the substrate is disposed over each light emitting element. Light emitted from the light flux controlling member is diffused by the light diffusion member so as to irradiate an illumination member (e.g., a liquid crystal panel) in a planar fashion (see, for example, PTL 1).

PTL 1 discloses a backlight module including a housing, a reflection plate with an upward inclination along a gentle curve in the height direction in the housing, and a plurality of light-emitting diode lenses (light flux controlling members) each including a light-emitting diode chip. The light-emitting diode lens includes a slender bottom surface, a pair of substantially semicircular reflection surfaces extended upward from the bottom surface, and an emission surface connecting the bottom surface and the reflection surface. This light-emitting diode lens is disposed to laterally emit light. The light emitted from the light-emitting diode lens is reflected by the reflection plate toward the LCD panel disposed on the upper side of the housing. At this time, the light emitted from the light-emitting diode lens uniformly irradiates the LCD panel because the reflection plate is gently tilted.

### CITATION LIST

### Patent Literature

PTL 1
Japanese Patent Application Laid-Open No. 2006-54407

### SUMMARY OF INVENTION

### Technical Problem

It is conceivable to irradiate a slender irradiated surface by using a light-emitting diode chip and a light-emitting diode lens as those disclosed in disclosed in PTL 1. In this case, the light-emitting diode lens is disposed with its major axis set along the major axis of the irradiated surface. When light is applied to the slender irradiated surface by using a light-emitting diode lens as that disclosed in disclosed in PTL 1, however, the light may reach also the region other than the irradiated surface. In addition, in the case where the installation space for the irradiated surface and the light-emitting diode lens is limited, the irradiated surface and the light-emitting diode lens are disposed close to each other, and consequently the irradiated surface may not be uniformly irradiated.

In view of this, an object of the present invention is to provide a light flux controlling member and an irradiation device that can uniformly irradiate the irradiated surface even when the irradiated surface is irradiated from a short distance, and can prevent light from arriving at regions other than the region of the irradiated surface. In addition, another object of the present invention is to provide a light emitting device including the light flux controlling member, and a sterilization device including the irradiation device.

### Solution to Problem

[1] A light flux controlling member configured to control light emitted from a light emitting element so as to spread the light when the light flux controlling member is disposed over the light emitting element so as to intersect an optical axis of the light emitting element, the light flux controlling member comprising: an incidence surface that is an inner surface of a recess that is open on a rear side, the incidence surface being configured to allow incidence of the light emitted from the light emitting element; two total reflection surfaces disposed on a front side, and configured to reflect, away from the optical axis in a different direction, a part of light entered from the incidence surface; two first emission surfaces configured to emit light reflected by the total reflection surface, to outside toward two directions along a first axis perpendicular to the optical axis; and a second emission surface disposed at a part on a second axis perpendicular to the optical axis and the first axis between the two total reflection surfaces, the second emission surface being configured to emit another part of the light entered from the incidence surface, to the outside while spreading the light, wherein the two total reflection surfaces are each a part of a surface that is obtained through rotation with a virtual half straight line as a rotation axis, wherein the two virtual half straight lines each intersect the first emission surface, wherein in plan view of the light flux controlling member, one ends of the two virtual half straight lines are located at the same point on the second axis, and an angle between the two virtual half straight lines is smaller than 180°, and wherein in plan view of the light flux controlling member, the angle between the two virtual half straight lines is formed on a side on which an area of the second emission surface is larger with respect to the first axis.

[2] The light flux controlling member according to [1], wherein the incidence surface includes: a first incidence surface disposed along the first axis to face the light emitting element; a second incidence surface that intersects the first axis; and a third incidence surface connecting the first incidence surface and an opening edge of the recess, and disposed at a position corresponding to the second emission surface, on one side with respect to the optical axis in a direction along the second axis, wherein the first incidence surface allows the light emitted from the light emitting element to enter toward the total reflection surface or the first emission surface, wherein the second incidence surface allows the light emitted from the light emitting element to enter toward the first emission surface, and wherein the third incidence surface allows the light emitted from the light emitting element to enter toward the second emission surface.

[3] The light flux controlling member according to [1] or [2], wherein in plan view of the light flux controlling member, the two virtual half straight lines are line-symmetric with respect to the second axis as a symmetry axis.

[4] A light emitting device comprising: a light emitting element; and the light flux controlling member according to any one of [1] to [3], wherein when, in an XYZ coordinate system with three axes orthogonal to each other, the light emitting element is disposed such that the optical axis of the light emitting element coincides with a Z axis, and that a center of a light-emitting surface of the light emitting element is located at an origin of the XYZ coordinate system, and the light flux controlling member is disposed such that the first axis coincides with an X axis, and that the side on which the area of the second emission surface area is larger with respect to the X axis is located on a plus side in a Y axis, the two virtual half straight lines each pass through the following coordinates (XA, YA, ZA) and (XB, YB, ZB) that satisfy Condition (1) or Condition (2):

XA>0,YA>0,ZA>0, and XB<0,YB>0,ZB>0, and (Condition1)

XA>0,YA>0,ZA<0, and XB<0,YB>0,ZB<0. (Condition2)

[5] An irradiation device comprising: a light emitting device including a light emitting element and a light flux controlling member configured to spread light emitted from the light emitting element; and an irradiated surface including a major axis and a minor axis, and configured to be irradiated with the light emitted from the light emitting device, wherein when, in an XYZ coordinate system with three axes orthogonal to each other, the light emitting element is disposed such that the optical axis of the light emitting element coincides with a Z axis, and that a center of a light-emitting surface of the light emitting element is located at an origin of the XYZ coordinate system, and the irradiated surface is disposed in an orientation of receiving the light emitted from the light emitting device so as to intersect the Z axis on a plus side in the Z axis with an X axis and the major axis being parallel to each other, the light flux controlling member is symmetric about a YZ cross-section including a Y axis and the Z axis, and the irradiation device has a luminance distribution with peaks in three directions toward points (Xn, Yn, Zn; n=1, 2 or 3) that satisfy the following conditions:

X1>0,Y1>0,Z1>0 and X1>Y1,X1>Z1 direction (Direction 1)

X2<0,Y2>0,Z2>0 and |X2|>Y2,|X2|>Z2 direction, and (Direction 2)

X3=0,Y3>0 and Z3>0 direction. (Direction 3)

[6] The irradiation device according to [5], wherein a shortest distance between the light emitting element and the irradiated surface is shorter than the minor axis.

[7] The irradiation device according to [5], wherein a center position in the minor axis of the irradiated surface is disposed on a plus side in the Y axis.

[8] The irradiation device according to any one of [5] to [7], wherein a ratio of a length of the major axis and a length of the minor axis is 4:1 to 8:1, and wherein in an XZ cross-section including the X axis and the Z axis, an angle between the optical axis and a straight line connecting the origin and an end portion of the irradiated surface is equal to or greater than 80° and smaller than 90°.

[9] The irradiation device according to any one of [5] to [8}, wherein the light flux controlling member includes: an incidence surface that is an inner surface of a recess that is open on a rear side, the incidence surface being configured to allow incidence of the light emitted from the light emitting element; two total reflection surfaces disposed on a front side, and configured to reflect, away from the optical axis in a different direction, a part of light entered from the incidence surface; two first emission surfaces configured to emit light reflected by the total reflection surface, to outside toward two directions along a first axis perpendicular to the optical axis; and a second emission surface disposed at a part on a second axis perpendicular to the optical axis and the first axis between the two total reflection surfaces, the second emission surface being configured to emit another part of the light entered from the incidence surface, to the outside while spreading the light, wherein the two total reflection surfaces are each a part of a surface that is obtained through rotation with a virtual half straight line as a rotation axis, wherein the two virtual half straight lines each intersect the first emission surface, wherein in plan view of the light flux controlling member, one ends of the two virtual half straight lines are located at the same point on the second axis, and an angle between the two virtual half straight lines is smaller than 180°, and wherein in plan view of the light flux controlling member, the angle between the two virtual half straight lines is formed on a side on which an area of the second emission surface is larger with respect to the first axis.

The irradiation device according to any one of [5] to [9}, wherein the light emitted from the light emitting element is an ultraviolet ray.

A sterilization device comprising the irradiation device according to any one of [5] to [10].

### Advantageous Effects of Invention

According to the present invention, it is possible to uniformly irradiate the irradiated surface even when the irradiated surface is irradiated from a short distance, and prevent light from arriving at regions other than the region of the irradiated surface.

### BRIEF DESCRIPTION OF DRAWINGS

FIGS. 1A to 1C are diagrams illustrating a configuration of an irradiation device of Embodiment 1;

FIGS. 2A to 2D are diagrams illustrating a configuration of a light flux controlling member of Embodiment 1;

FIGS. 3A to 3D are sectional views of the light flux controlling member of Embodiment 1;

FIGS. 4A to 4C are diagrams for describing a virtual half straight line;

FIGS. 5A and 5B are diagrams illustrating light paths of the light emitting device;

FIGS. 6A to 6E are diagrams illustrating an illuminance distribution in an irradiated surface;

FIGS. 7A and 7B are diagrams for describing a luminous intensity distribution of light emitted from the light emitting device;

FIGS. 8A and 8B are diagrams for describing a luminous intensity distribution of light emitted from the light emitting device;

FIGS. 9A and 9B are diagrams for describing a luminous intensity distribution of light emitted from the light emitting device;

FIGS. 10A to 10C are diagrams for describing a luminous intensity distribution of light emitted from the light emitting device;

FIGS. 11A to 11D are diagrams illustrating a configuration of a light flux controlling member of Embodiment 2;

FIGS. 12A to 12D are sectional views of the light flux controlling member of Embodiment 2;

FIGS. 13A to 13C are diagrams for describing a virtual half straight line;

FIGS. 14A and 14B are diagrams illustrating light paths of the light emitting device; and

FIGS. 15A to 15E are diagrams illustrating an illuminance distribution in an irradiated surface.

### DESCRIPTION OF EMBODIMENTS

Irradiation devices according to embodiments of the present invention are elaborated below with reference to the accompanying drawings.

### Embodiment 1

### Configuration of Irradiation Device

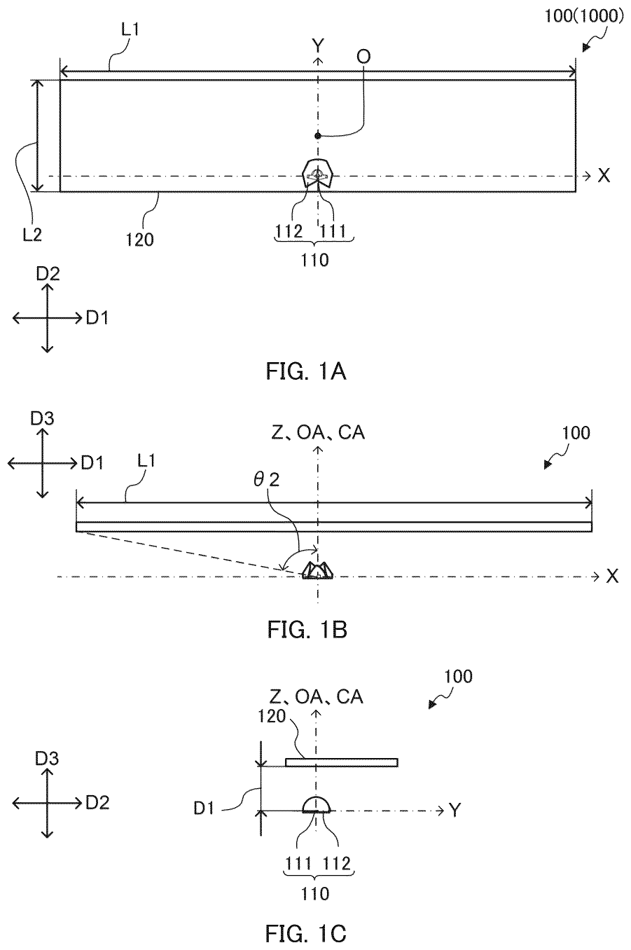

FIG. 1A is a bottom view of irradiation device 100 of Embodiment 1, FIG. 1B is a front view, and FIG. 1C is a left side view.

As illustrated in FIGS. 1A to 1C, irradiation device 100 includes light emitting device 110 including light emitting element 111 and light flux controlling member 112, and irradiated surface 120. In the case where light emitted from light emitting device 110 is an ultraviolet ray, irradiation device 100 can function as sterilization device (1000).

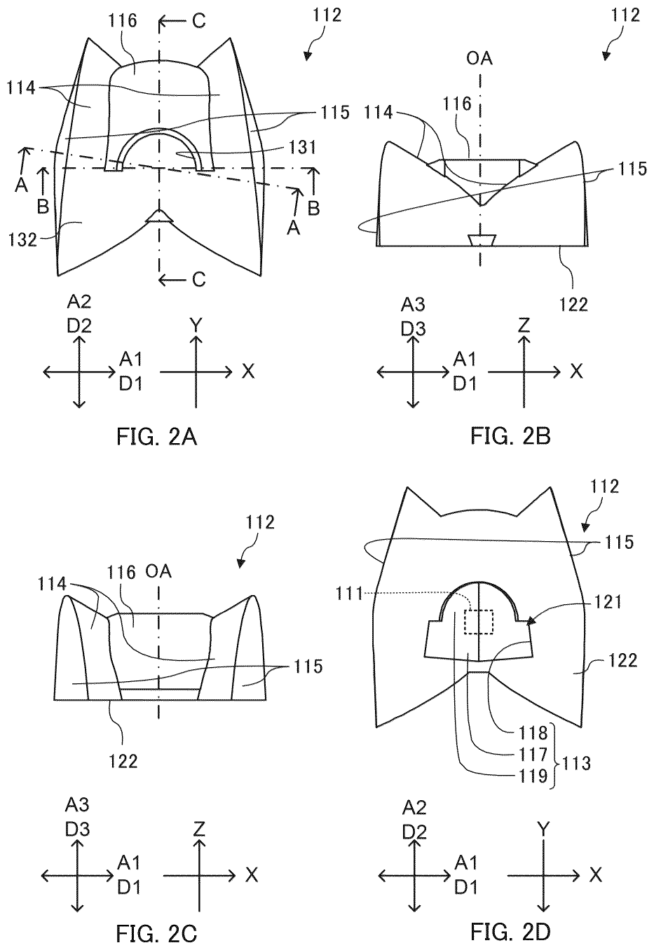

Light emitting device 110 is disposed at a predetermined position with respect to irradiated surface 120, and emits light to irradiated surface 120. In the present embodiment, light emitting device 110 is fixed to a holder not illustrated in the drawings. Light emitting device 110 includes light emitting element 111, and light flux controlling member 112. The number of light emitting device 110 is not limited. One or a plurality of light emitting devices 110 may be provided. In the present embodiment, one light emitting device 110 is provided. In addition, in the present embodiment, optical axis OA of light emitting element 111 and central axis CA of light flux controlling member 112 coincide with each other. Note that optical axis OA of light emitting element 111 means the central light beam of a three-dimensional emission light flux from light emitting element 111. In addition, central axis CA of light flux controlling member 112 is parallel to third axis D3, and passes through the center of first axis A1 in incidence surface 113 and through the center of second axis A2 in incidence surface 113 (see FIG. 2D). The positional relationship between light emitting device 110 and irradiated surface 120 is described later.

Light emitting element 111 emits light with a predetermined wavelength. The type of light emitting element 111 is not limited as long as predetermined light can be emitted. Light emitted from light emitting element 111 may be a visible light ray or an ultraviolet ray. Examples of light emitting element 111 include a light-emitting diode (LED), a mercury lamp, a metal halide lamp, a xenon lamp, and a laser diode (LD). When an ultraviolet ray is emitted from light emitting element 111, the central wavelength or peak wavelength of the ultraviolet ray is preferably 200 nm or greater and 350 nm or smaller. The central wavelength or peak wavelength of the ultraviolet ray emitted from light emitting element 111 is more preferably 250 nm or greater and 290 nm or smaller from a view point of sterilization efficiency. That is, the ultraviolet ray is more preferably ultraviolet ray C waves (UVC).

### Configuration of Light Flux Controlling Member

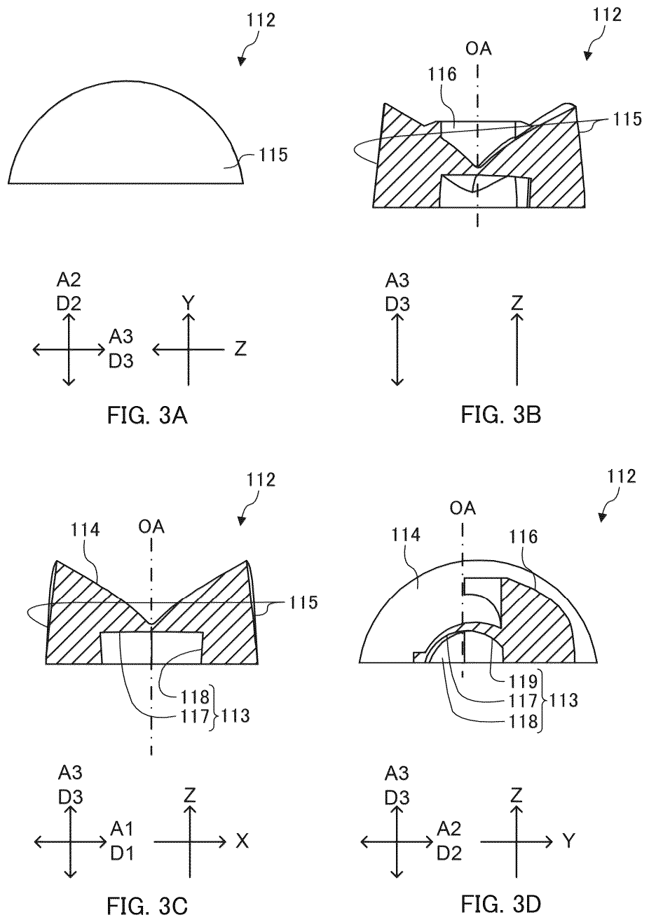

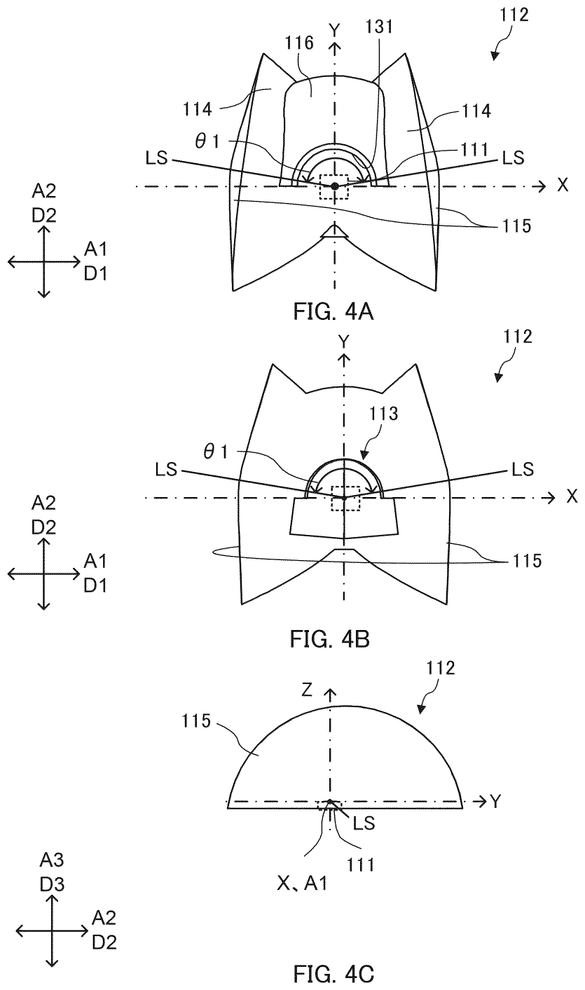

FIG. 2A is a plan view of light flux controlling member 112, FIG. 2B is a front view, FIG. 2C is a rear view, and FIG. 2D is a bottom view. FIG. 3A is a right side view of light flux controlling member 112, FIG. 3B is a sectional view taken along line A-A of FIG. 2A, FIG. 3C is a sectional view taken along line B-B of FIG. 2A, and FIG. 3D is a sectional view taken along line C-C of FIG. 2A. FIG. 4A is a plan view of light flux controlling member 112 for describing virtual half straight line LS, FIG. 4B is a bottom view of light flux controlling member 112 for describing virtual half straight line LS, and FIG. 4C is a right side view of light flux controlling member 112 for describing virtual half straight line LS. In the following description, the two directions along first axis A1 perpendicular to optical axis OA of light emitting element 111 are X direction D1, the two directions along second axis A2 perpendicular to first axis A1 in plan view of light emitting device 110 are Y direction D2, and the two directions that are perpendicular to first axis A1 and second axis A2 and are along third axis A3 are Z direction D3. In addition, in the XYZ coordinate system with the three axes orthogonal to each other, X direction D1 is parallel to the X axis, Y direction D2 is parallel to the Y axis, and Z direction is parallel to the Z axis. Note that in the present embodiment, first axis A1 coincides with the X axis, second axis A2 coincides with the Y axis, and third axis A3 coincides with optical axis OA and the Z axis.

Light flux controlling member 112 is a member that controls light emitted from light emitting element 111 to spread the light when disposed over light emitting element 111 to intersect optical axis OA of light emitting element 111. As illustrated in FIGS. 2A to 2D and FIGS. 3A to 3D, light flux controlling member 112 includes incidence surface 113, two total reflection surfaces 114, two first emission surfaces 115, and second emission surface 116. In the present embodiment, light flux controlling member 112 has a shape that is plane symmetrical with respect to a plane (YZ plane) including optical axis OA (Z axis) and second axis A2 (Y axis).

Incidence surface 113 is an inner surface of a recess that is open on the rear side, and allows light emitted from light emitting element 111 to enter light flux controlling member 112. The configuration of incidence surface 113 is not limited. In the present embodiment, incidence surface 113 includes first incidence surface 117, second incidence surface 118, and third incidence surface 119.

First incidence surface 117 is disposed along first axis A1 to face light emitting element 111. As used herein, the direction along first axis A1 may be parallel to first axis A1, and may be tilted within a range of ±20° with respect to first axis A1. In the present embodiment, first incidence surface 117 is composed of two straight lines in a cross-section taken along a plane (XZ plane) including optical axis OA and first axis A1 (X axis) (see FIG. 3C). More specifically, it is configured to come closer to the front side with decreasing distance to optical axis OA in X direction D1. In addition, first incidence surface 117 is formed in a curved line in a cross-section taken along a plane (YZ plane) including optical axis OA (Z axis) and second axis A2 (Y axis) (see FIG. 3D). First incidence surface 117 allows light emitted from light emitting element 111 to enter toward total reflection surface 114 or first emission surface 115.

Second incidence surface 118 is disposed along optical axis OA to connect first incidence surface 117 and the opening edge of recess 121. Here, the direction along optical axis OA may be parallel to optical axis OA, or may be tilted within a range of ±5° with respect to optical axis OA. More specifically, in the present embodiment, second incidence surface 118 is tilted so as to come close to optical axis OA (third axis D3) as it goes from the bottom surface of recess 121 toward the opening edge of recess 121 (see FIG. 3C). Second incidence surface 118 allows light emitted from light emitting element 111 to enter toward first emission surface 115.

Third incidence surface 119 is disposed on one side with respect to optical axis OA in the direction (Y direction D2) along second axis A2, at a position corresponding to second emission surface 116, so as to connect first incidence surface 117 and the opening edge of recess 121. Third incidence surface 119 is formed in a curved line in a cross-section taken along a plane (XZ plane) including optical axis OA (Z axis) and first axis A1 (X axis). In addition, third incidence surface 119 is formed in a curved line in a cross-section taken along a plane (YZ plane) including optical axis OA (Z axis) and second axis A2. More specifically, third incidence surface 119 is formed in a substantially hemispheric shape (see FIGS. 2D and 3D). Third incidence surface 119 allows light emitted from light emitting element 111 to enter toward second emission surface 116.

Two total reflection surfaces 114 are disposed on the front side to reflect a part of the light entered from incidence surface 113, to go away from optical axis OA in a different direction. In the present embodiment, two total reflection surfaces 114 are disposed on the front side, and reflect the light entered from incidence surface 113 such that the light goes away from optical axis OA toward the two directions (X direction D1) along first axis A1 perpendicular to optical axis OA. Specifically, two total reflection surfaces 114 reflect the arriving light toward two first emission surfaces 115.

In a cross-section taken along a plane (XZ plane) including first axis A1 (X axis) and third axis A3 (Z axis), total reflection surface 114 is formed such that the height from rear surface 122 increases from the center toward the both end portions with central axis CA located at the center. More specifically, in a cross-section taken along a plane (XZ plane) including first axis A1 (X axis) and third axis A3 (Z axis), total reflection surfaces 114 are each formed such that the gradient of the tangent gradually decreases as it goes from the center toward the end portion. Note that total reflection surface 114 is preferably designed such that the light in the direction along the third axis (Z axis) is totally reflected.

As illustrated in FIGS. 4A to 4C, two total reflection surfaces 114 are each a part of a surface obtained by rotating a curved line with virtual half straight line LS as a rotation axis. That is, in the present embodiment, two virtual half straight lines LS are provided. In plan view of light flux controlling member 112, one ends of two virtual half straight lines LS are located at the same point on second axis A2, and the angle between two virtual half straight lines LS is smaller than 180°. Each of two virtual half straight lines LS intersects first emission surface 115. More specifically, the virtual half straight line on the left side in FIGS. 4A and 4B intersects first emission surface 115 on the left side in FIGS. 4A and 4B, and the virtual half straight line on the right side in FIGS. 4A and 4B intersects first emission surface 115 on the right side in FIGS. 4A and 4B. That is, each of the two virtual half straight lines intersects corresponding first emission surface 115 with respect to corresponding second axis A2 (Y axis).

The position of one end of virtual half straight line LS on second axis A2 is not limited. The position of one end of virtual half straight line LS on second axis A2 may be disposed on the plus side, minus side or origin of second axis A2 (Y axis). In the present embodiment, the position of one end of virtual half straight line LS on second axis A2 is the same as the position of the origin in XYZ coordinate system.

Angle θ1 between two line segments LS is formed on the side on which the area of second emission surface 116 is larger. Here, the side on which the area of second emission surface 116 is larger means the side on which the area of second emission surface 116 is larger with respect to first axis A1 (X axis) in plan view of light flux controlling member 112. In the present embodiment, second emission surface 116 is disposed only on one of side (the plus side in the Y axis) of second axis A2 with respect to first axis A1 (X axis). Thus, in the present embodiment, the side on which the area of second emission surface 116 is larger means one side of the second axis (the plus side in the Y axis) with respect to first axis A1 (X axis).

Two virtual half straight lines LS are line-symmetric with respect to second axis A2 as a symmetry axis in plan view of light flux controlling member 112. Specifically, the angle between optical axis OA and one virtual half straight line LS and the angle between optical axis OA and the other virtual half straight line LS are the same angle.

In addition, virtual half straight line LS can also be said to pass through the following point described below. As illustrated in FIGS. 4A to 4C, light emitting element 111 is disposed such that in the XYZ coordinate system with the three axes orthogonal to each other, optical axis OA of light emitting element 111 coincides with the Z axis, and that the center of the light-emitting surface of light emitting element 111 is located at the origin of the XYZ coordinate system. When light flux controlling member 112 is disposed such that first axis A1 coincides with the X axis and that the side on which the area of second emission surface 116 is larger is located on the plus side in the Y axis, two virtual half straight lines LS each pass through the following coordinates (XA, YA, ZA) and (XB, YB, ZB) that satisfy Condition (1) or Condition (2).

XA>0,YA>0,ZA>0, and XB<0,YB>0,ZB>0 (Condition1)

XA>0,YA>0,ZA<0, and XB<0,YB>0,ZB<0 (Condition2)
In the present embodiment, virtual half straight line LS satisfies Condition (2).

Two first emission surfaces 115 emits at least a part (main light) of light reflected by total reflection surface 114, to the outside toward two directions (X direction D1) along first axis A1 perpendicular to optical axis OA(X axis). Two first emission surfaces 115 are disposed with two total reflection surfaces 114 sandwiched therebetween so as to emit, to the outside, light reflected by total reflection surface 114. The configuration of first emission surface 115 is not limited as long as the above-mentioned function can be ensured. First emission surface 115 may be composed of one surface or a plurality of surfaces. In the present embodiment, first emission surface 115 is composed of one curved surface. More specifically, first emission surface 115 may have a circular shape. In the present embodiment, first emission surface 115 is formed as a side surface of a cone. In addition, first emission surface 115 is disposed such that the central axis of the cone is tilted with respect to first axis A1. Note that the central axis of the cone may coincide with virtual half straight line LS. The inclination angle of the central axis of the cone with respect to first axis A1 is not limited as long as light emitted from first emission surface 115 can be efficiently emitted in the longitudinal axial direction of irradiated surface 120. In this manner, a part of light emitted from light emitting element 111 can be appropriately applied in the longitudinal axial direction of irradiated surface 120 even in the case where light emitting element 111 and irradiated surface 120 are close to each other. Here, the main light means light of 90% or more of the light reflected by total reflection surface 114 set as 100%. Note that preferably, first emission surface 115 emits all of the light reflected by total reflection surface 114.

Second emission surface 116 is disposed at a portion on second axis A2 perpendicular to optical axis OA and first axis A1 between two total reflection surfaces 114 so as to emit the other part of the light entered from incidence surface 113, to the outside while spreading the light. In the present embodiment, second emission surface 116 spreads the emitted light in the YZ cross-section and the XZ cross-section. The configuration of second emission surface 116 is not limited as long as the above-mentioned function can be ensured. Second emission surface 116 may be composed of one surface or a plurality of surfaces. In the present embodiment, second emission surface 116 is composed of one curved surface. Second emission surface 116 is formed in a curved line in a cross-section taken along a plane (YZ plane) including optical axis OA (Z axis) and second axis D2 (Y axis). The size of second emission surface 116 relative to total reflection surface 114 is set as necessary in accordance with irradiated surface 120. In the present embodiment, second emission surface 116 includes notch part 131. Notch part 131 is disposed on the front side of light flux controlling member 112. Notch part 131 adjusts the quantity of light reaching total reflection surface 114 and the quantity of light reaching second emission surface 116. The size of notch part 131 is set as necessary based on the above-mentioned function.

Irradiated surface 120 is irradiated with light by light emitting device 110. The size of irradiated surface 120 is not limited. The size of irradiated surface 120 is set as necessary. The shape of irradiated surface 120 is also not limited. In the present embodiment, irradiated surface 120 includes major axis L1 and minor axis L2. The “major axis L1” means the longest line segment in plan view of irradiated surface 120. The “minor axis L2” means the shortest line segment in plan view of irradiated surface 120. Irradiated surface 120 may be a flat surface or a curved surface. Preferably, the ratio of the lengths of the major axis and the length of the minor axis L2 is 4:1 to 8:1.

Now a positional relationship between irradiated surface 120 and light emitting device 110 is described below with reference to FIG. 1. As illustrated in FIGS. 1A to 1C, irradiation device 100 are disposed as follows in the XYZ coordinate system with the three axes (the X axis, the Y axis and the Z axis) orthogonal to each other.

As illustrated in FIGS. 1A to 1C, light emitting element 111 is disposed such that optical axis OA of light emitting element 111 coincides with the Z axis, and that the center of the light-emitting surface of light emitting element 111 is located at the origin of the XYZ coordinate system. Light flux controlling member 112 is disposed such that it is symmetric about a plane (YZ plane) including second axis A2 (Y axis) and third axis A3 (Z axis), that first axis A1 is parallel to the X axis, and that the side on which the area of second emission surface 116 is larger is located on the plus side in the Y axis. Irradiated surface 100 is disposed in an orientation of receiving light emitted from light emitting device 110 such that the center position in its minor axis intersects the Z axis on the plus side in the Z axis and the plus side in the Y axis, and that the X axis and major axis L1 are parallel to each other.

Preferably, the shortest distance D1 of light emitting element 111 and irradiated surface 120 is smaller than minor axis L2. In a cross-section taken along a plane (XZ cross-section) including first axis A1 (X axis) and third axis A3 (Z axis), angle θ2 between optical axis OA and a straight line connecting the origin of the XYZ coordinate system and the end portion of irradiated surface 120 is preferably equal to or greater than 80° and smaller than 90°. In this manner, light emitting element 111 and irradiated surface 120 are disposed close to each other.

### Light Path Diagram and Illuminance Distribution

Now a light path diagram of light emitting device 110 and an illuminance distribution at irradiated surface 120 of irradiation device 100 are described below. Here, the ratio of the length of major axis L1 of irradiated surface 120, the length of minor axis L2, and distance D1 of the light-emitting surface of light emitting element 111 and irradiated surface 120 is 43.3:6.6:1. In addition, the ratio of the length of minor axis L2, and the length of the center of minor axis L2 and optical axis OA of light emitting element 111 is 20:7.

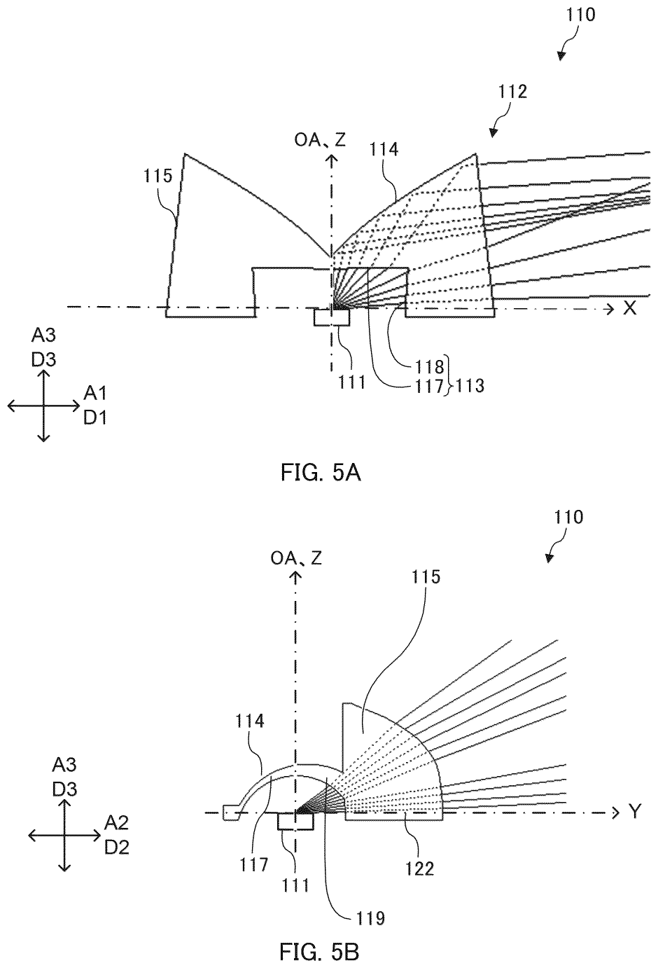

FIG. 5A is a diagram illustrating light paths of light entered from first incidence surface 117 and second incidence surface 118 in a cross-section taken along a plane (XZ plane) including first axis A1 (X axis) and third axis A3 (Z axis), and FIG. 5B is a diagram illustrating light paths of light entered from third incidence surface 119 in a cross-section taken along a plane (YZ plane) including second axis A2 (Y axis) and third axis A3 (Z axis). Note that FIG. 5A illustrates only light emitted toward the left half with respect to optical axis OA in a cross-section taken along the XZ plane, and FIG. 5B illustrates only light emitted toward the right half with respect to optical axis OA in a cross-section taken along the YZ plane.

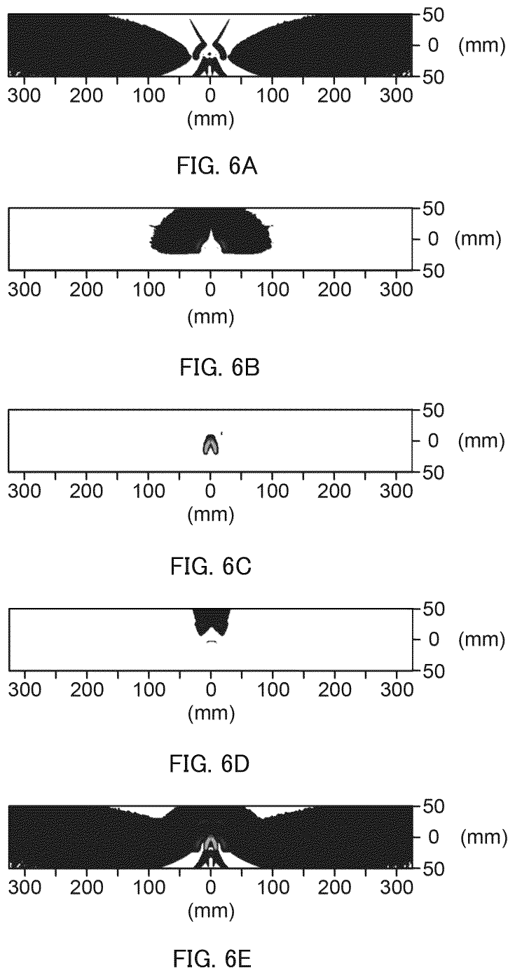

FIGS. 6A to 6E are diagrams illustrating an illuminance distribution of light in irradiated surface 120. FIG. 6A illustrates an illuminance distribution of light that is entered from first incidence surface 117 or second incidence surface 118 and is emitted from first emission surface 115, FIG. 6B illustrates an illuminance distribution of light that is entered from third incidence surface 119 and is emitted from first emission surface 115, FIG. 6C illustrates an illuminance distribution of light that is entered from first incidence surface 117 or second incidence surface 118 and is emitted from second emission surface 116, FIG. 6D illustrates an illuminance distribution of light that is entered from third incidence surface 119 and is emitted from second emission surface 116, and FIG. 6E illustrates an illuminance distribution of light that is entered from first incidence surface 117 or second incidence surface 118 and is emitted from first emission surface 115 and second emission surface 116. In FIGS. 6A to 6E, the abscissa indicates the distance from the center of major axis L1, and the ordinate indicates the distance from the center of minor axis L2.

As illustrated in FIGS. 5A and 6A, in a cross-section taken along a plane (XZ plane) including first axis A1 (X axis) and third axis A3 (Z axis), light emitted from the light emission center of light emitting element 111 with a small angle (emission angle) with respect to the optical axis of light emitting element 111 enters light flux controlling member 112 from first incidence surface 117. The light entered from first incidence surface 117 with a small angle with respect to optical axis OA reaches total reflection surface 114. The light having reached total reflection surface 114 is internally reflected toward first emission surface 115. The light reflected by total reflection surface 114 is emitted to the outside from first emission surface 115. In addition, as illustrated in FIG. 5A, the light entered from first incidence surface 117 with a large angle with respect to optical axis OA is emitted to the outside from first emission surface 115 without reaching total reflection surface 114.

As illustrated in FIGS. 5A and 6A, in a cross-section taken along a plane (XZ plane) including first axis A1 (X axis) and third axis A3 (Z axis), light emitted from the light emission center of light emitting element 111 with a further larger angle (emission angle) with respect to the optical axis of light emitting element 111 enters light flux controlling member 112 from second incidence surface 118. The light entered from second incidence surface 118 is emitted from first emission surface 115. At this time, in a cross-section taken along the XZ plane, the light that is emitted on the rear side (rear surface 122 side) than the vertex of second emission surface 116 is refracted toward the upper side. On the other hand, in a cross-section taken along the XZ plane, the light that is emitted on the front side than the vertex of second emission surface 116 advances while being slightly focused.

As illustrated in FIGS. 5B and 6D, in a cross-section taken along the YZ plane, a part of the light that is emitted from the light emission center of light emitting element 111 enters light flux controlling member 112 from third incidence surface 119. Light entered from third incidence surface 119 is emitted in a spreading manner at second emission surface 116. More specifically, light entered from third incidence surface 119 is spread only on the plus side in Y direction D2. Since irradiated surface 120 is disposed on the minus side in Y direction D2, the light entered from third incidence surface 119 can be applied to Y direction D2 of irradiated surface 120. In addition, in a cross-section taken along the XZ plane, it is spread to the plus side and minus side in X direction D1. Note that in FIG. 5B, in a cross-section taken along the YZ plane, light that is emitted from the center of the light-emitting surface of light emitting element 111 and is emitted toward the left half with respect to optical axis OA is totally reflected by total reflection surface 114, and is therefore not illustrated.

In addition, as illustrated in FIG. 6B, a part of the light that is entered from third incidence surface 119 is emitted from first emission surface 115. In addition, as illustrated in FIG. 6C, a part of the light that is entered from first incidence surface 117 or second incidence surface 118 is emitted from second emission surface 116.

As illustrated in FIG. 6E, the light that is entered from first incidence surface 117 or second incidence surface 118 and is emitted from first emission surface 115 and second emission surface 116 can be applied to the entirety of irradiated surface 120.

### Luminous Intensity Distribution

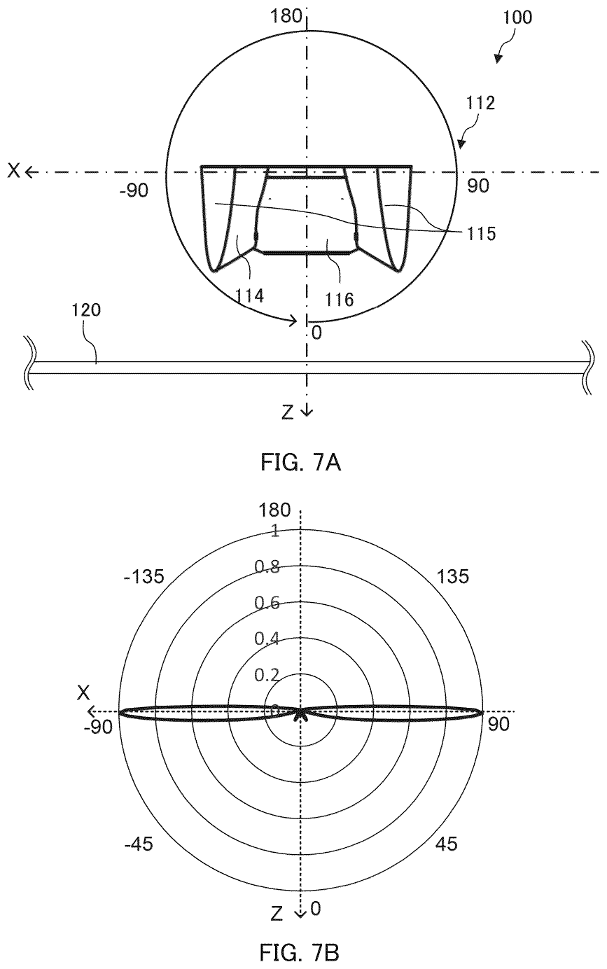

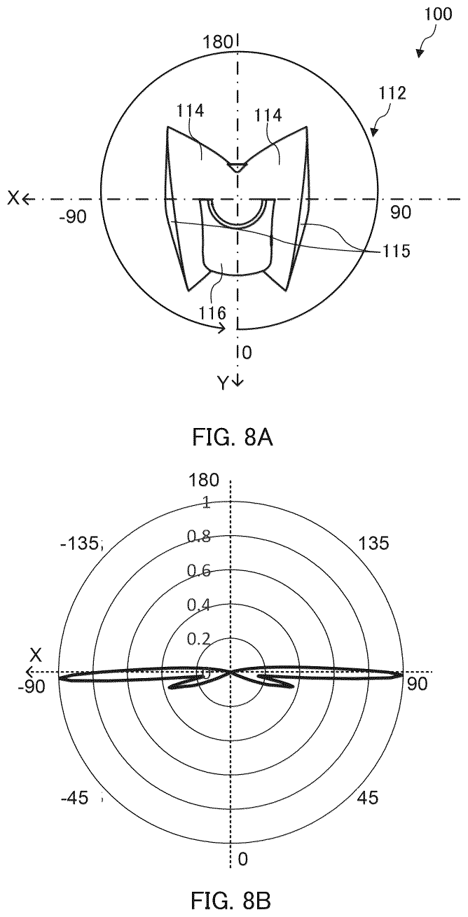

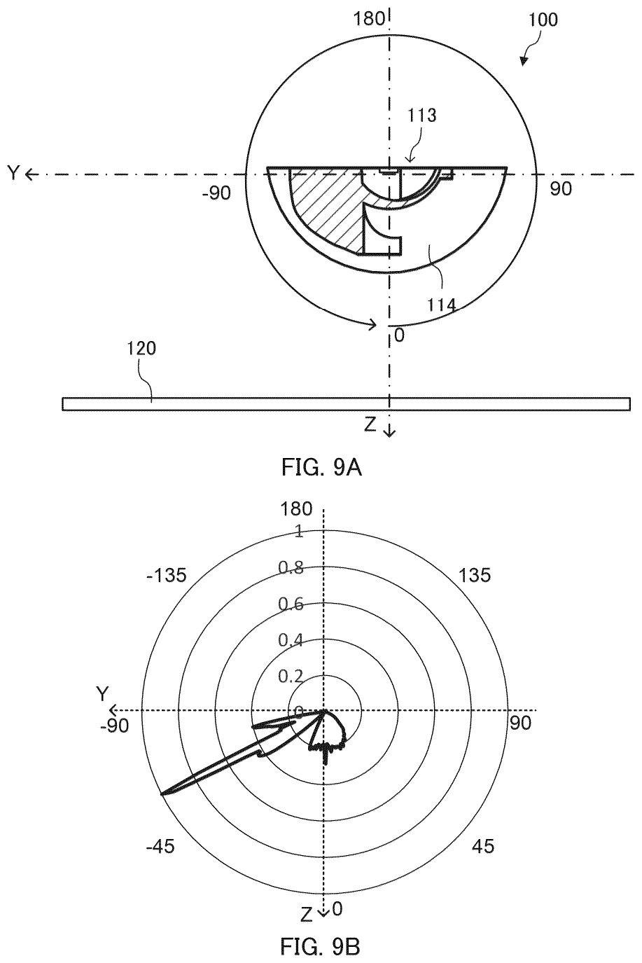

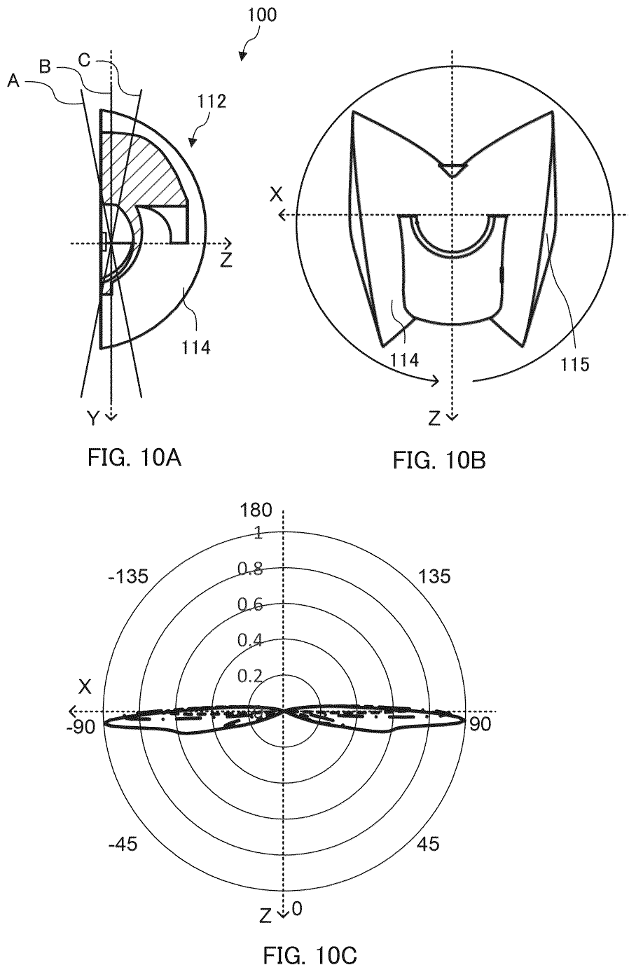

The luminous intensity distribution in irradiation device 100 was simulated. FIG. 7A is a schematic view of irradiation device 100 illustrating a measurement direction of a luminous intensity, and FIG. 7B illustrates a simulation result. FIG. 8A is a schematic view of irradiation device 100 illustrating a measurement direction of a luminous intensity, and FIG. 8B illustrates a simulation result. FIG. 9A is a schematic view of irradiation device 100 illustrating a measurement direction of a luminous intensity, and FIG. 9B illustrates a simulation result. FIGS. 10A and 10B are schematic views of irradiation device 100 illustrating a measurement direction of a luminous intensity, and FIG. 10C illustrates a simulation result.

As illustrated in FIGS. 7A and 7B, FIGS. 8A and 8B, FIGS. 9A and 9B and FIGS. 10A to 10C, a part of the light emitted from light emitting element 111 is emitted in a direction of ±90° in plan view of light flux controlling member 112. In addition, a part of the light emitted from light emitting element 111 is emitted in a direction of −60° in side view of irradiation device 100.

More specifically, when light emitting element 111 is disposed such that in the XYZ coordinate system with the three axes orthogonal to each other, optical axis OA of light emitting element 111 coincides with the Z axis, and that the center of the light-emitting surface of light emitting element 111 is located at the origin of the XYZ coordinate system, and irradiated surface 120 is disposed in an orientation of receiving light emitted from light emitting device 110 so as to intersect the Z axis on plus side in the Z axis with the X axis and major axis L1 being parallel to each other, a luminance distribution with peaks in three directions from light emitting device 110 toward points (Xn, Yn, Zn; n=1, 2 or 3) that satisfy the following conditions is provided.

X1>0,Y1>0,Z1>0 and X1>Y1,X1>Z1 direction (Direction 1)

X2<0,Y2>0,Z2>0 and |X2|>Y2,|X2|>Z2 direction (Direction 2)

X3≈0,Y3>0 and Z3>0 direction (Direction 3)
Here, X3≈0 means a range of ±5° with the direction along the Z axis set as 0° as viewed along the Y axis.

Here, the points (X1, Y1, Z1) and the points (X1, Y1, Z1) correspond to light emitted in a direction of +90° in FIGS. 7B and 8B. In addition, the points (X3, Y3, Z3) correspond to light emitted in a direction of −60° in FIG. 9B.

### Effects

As described above, in irradiation device 100 of the present embodiment, which is provided with first emission surface 115 and second emission surface 116 disposed on one side in the direction along second axis A2 between two first emission surfaces 115, irradiated surface 120 can be uniformly irradiated even when irradiated surface 120 is irradiated from a short distance, and light can be prevented from arriving at regions other than the region of irradiated surface 120.

### Embodiment 2

Next, an irradiation device according to Embodiment 2 is described. The irradiation device according to the present embodiment is different from irradiation device 100 of Embodiment 1 in the configuration of light flux controlling member 212. In view of this, here, the configuration of light flux controlling member 212 is mainly described. The same components as those of Embodiment 1 are denoted with the same reference numerals and the description thereof will be omitted.

### Configurations of Irradiation Device and Light Flux Controlling Member

The irradiation device according to the present embodiment includes light emitting device 210 including light emitting element 111 and light flux controlling member 212, and irradiated surface 120.

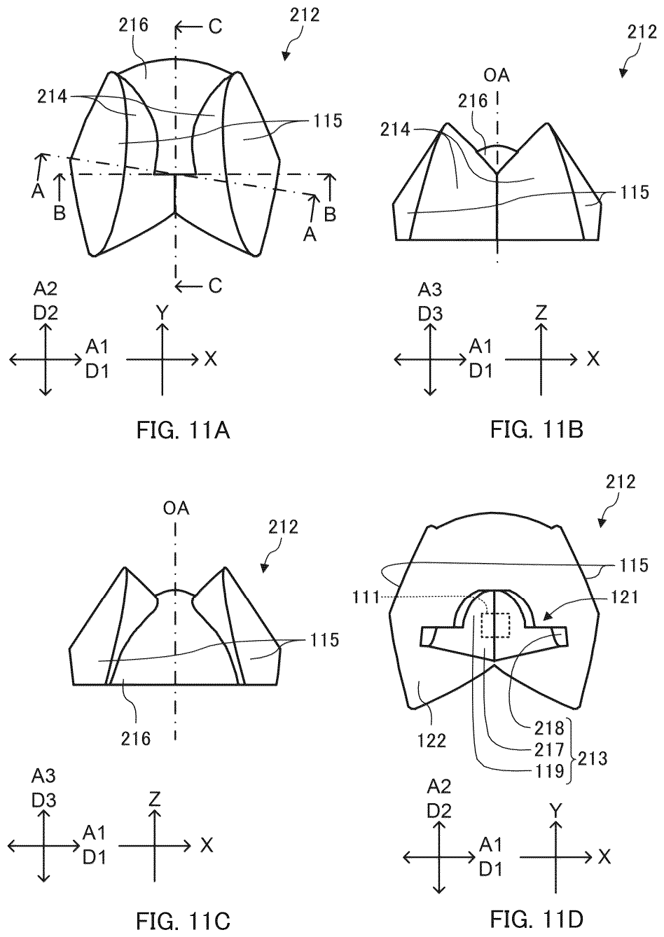

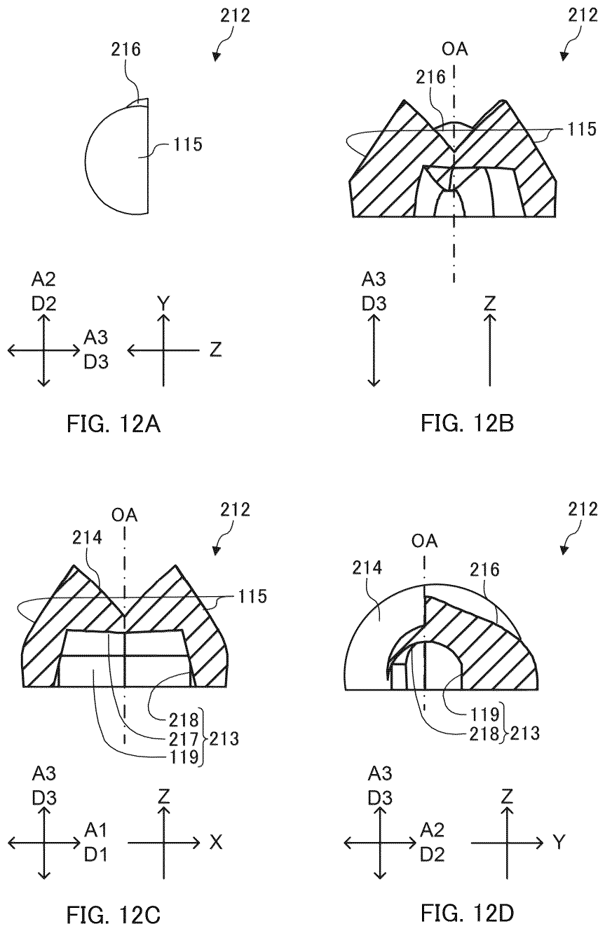

FIG. 11A is a plan view of light flux controlling member 212, FIG. 11B is a front view, FIG. 11C is a rear view, and FIG. 11D is a bottom view. FIG. 12A is a right side view of light flux controlling member 212, FIG. 12B is a sectional view taken along line A-A of FIG. 11A, FIG. 12C is a sectional view taken along line B-B of FIG. 11A, and FIG. 12D is a sectional view taken along line C-C of FIG. 11A.

As illustrated in FIGS. 11A to 11D and FIGS. 12A to 12D, light flux controlling member 212 includes incidence surface 213, two total reflection surfaces 214, two first emission surfaces 115, and second emission surface 216. Note that total reflection surface 214 and first emission surface 115 are the same as those of Embodiment 1, and therefore the description thereof will be omitted.

Incidence surface 213 includes first incidence surface 217, second incidence surface 218, and third incidence surface 119. In the present embodiment, first incidence surface 217 is formed in a substantially linear shape in a cross-section taken along a plane (XZ plane) including optical axis OA (Z axis) and first axis A1 (X axis). More specifically, first incidence surface 117 is tilted toward total reflection surface 114 side (toward the plus side in Z direction D3) as it goes away from optical axis OA in X direction D1 (see FIG. 12C).

Second incidence surface 218 is formed in a substantially linear shape in a cross-section taken along a plane (XZ plane) including optical axis OA (Z axis) and first axis A1 (X axis). More specifically, second incidence surface 218 is tilted toward rear surface 122 (toward the minus side in Z direction D3) as it goes away from optical axis OA in X direction D1 (see FIG. 12C).

In the present embodiment, second emission surface 216 does not include notch part 131.

In a cross-section taken along a plane (XZ plane) including first axis A1 (X axis) and third axis A3 (Z axis), total reflection surface 214 is formed such that the height from rear surface 122 increases from the center toward the both end portions with central axis CA located at the center.

More specifically, in a cross-section taken along a plane (XZ plane) including first axis A1 (X axis) and third axis A3 (Z axis), total reflection surface 214 is formed such that the gradient of the tangent gradually decreases as it goes from the center toward the end portion. Note that total reflection surface 214 is preferably designed such that the light in the direction along the third axis (Z axis) is totally reflected.

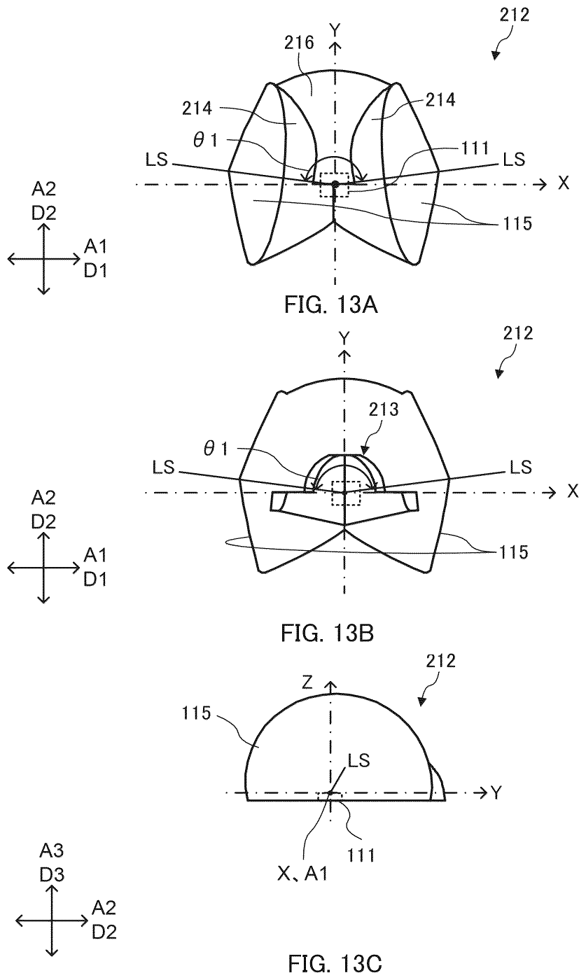

As illustrated in FIGS. 13A to 13C, two total reflection surfaces 214 are each a part of a surface obtained by rotating a curved line with virtual half straight line LS as a rotation axis. That is, in the present embodiment, two virtual half straight lines LS are provided. In plan view of light flux controlling member 112, one ends of two virtual half straight lines LS are located at the same point on second axis A2, and the angle between two virtual half straight lines LS is smaller than 180°.

The position of one end of virtual half straight line LS on second axis A2 is not limited. The position of one end of virtual half straight line LS on second axis A2 may be disposed on the plus side, minus side or origin of second axis A2 (Y axis). In the present embodiment, the position of one end of virtual half straight line LS on second axis A2 is the same as the position of the origin in XYZ coordinate system.

Angle θ1 between two line segments LS is formed on the side on which the area of second emission surface 216 is larger. Here, the side on which the area of second emission surface 216 is larger means the side on which the area of second emission surface 216 is larger with respect to first axis A1 (X axis) in plan view of light flux controlling member 112. In the present embodiment, second emission surface 216 is disposed only on one of side (the plus side in the Y axis) of second axis A2 with respect to first axis A1 (X axis). Thus, in the present embodiment, the side on which the area of the second emission surface 216 is larger means one side of the second axis (the plus side in the Y axis) with respect to first axis A1 (X axis).

Two virtual half straight lines LS are line-symmetric with respect to second axis A2 as a symmetry axis in plan view of light flux controlling member 212. Specifically, the angle between optical axis OA and one virtual half straight line LS and the angle between optical axis OA and the other virtual half straight line LS are the same angle.

In addition, virtual half straight line LS can also be said to pass through the following point described below. As illustrated in FIGS. 13A to 13C, light emitting element 111 is disposed such that in the XYZ coordinate system with the three axes orthogonal to each other, optical axis OA of light emitting element 111 coincides with the Z axis, and that the center of the light-emitting surface of light emitting element 111 is located at the origin of the XYZ coordinate system. When light flux controlling member 212 is disposed such that first axis A1 coincides with the X axis, and that the side on which the area of second emission surface 216 is larger is located on the plus side in the Y axis, two virtual half straight lines LS each pass through the following coordinates (XA, YA, ZA) and (XB, YB, ZB) that satisfy Condition (1) or Condition (2).

XA>0,YA>0,ZA>0, and XB<0,YB>0,ZB>0 (Condition1)

XA>0,YA>0,ZA<0, and XB<0,YB>0,ZB<0 (Condition2)
In the present embodiment, virtual half straight line LS satisfies Condition (1).

### Light Path Diagram and Illuminance Distribution

Now a light path diagram in light emitting device 210 and an illuminance distribution in irradiated surface 120 in the irradiation device are described below. Here, the ratio of the length of major axis L1 of irradiated surface 120, the length of minor axis L2, and distance D1 of the light-emitting surface of light emitting element 111 and irradiated surface 120 is 43.3:6.6:1. In addition, the ratio of the length of minor axis L2, and the length of the center of minor axis L2 and optical axis OA of light emitting element 111 is 20:7.

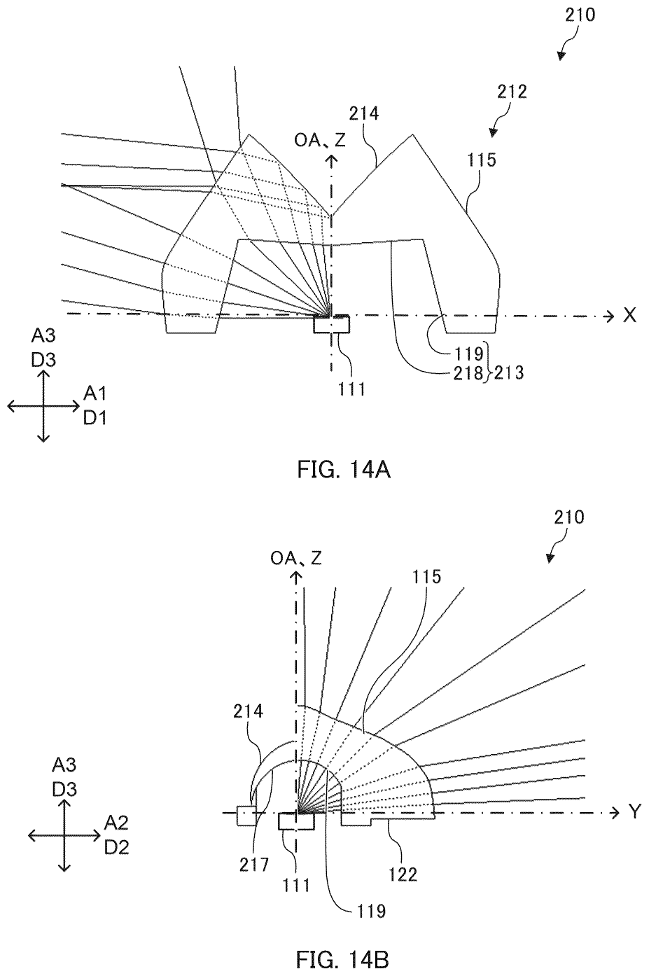

FIG. 14A is a diagram illustrating light paths of light entered from first incidence surface 217 and second incidence surface 118 in a cross-section taken along a plane (XZ plane) including optical axis OA and first axis A1 (X axis), and FIG. 14B is a diagram illustrating light paths of light entered from third incidence surface 119 in a cross-section taken along a plane (YZ plane) including optical axis OA and second axis A2 (Y axis). Note that FIG. 14A illustrates only light emitted toward the left half with respect to optical axis OA in a cross-section taken along the XZ plane, and FIG. 14B illustrates only light emitted toward the right half with respect to optical axis OA in a cross-section taken along the XZ plane.

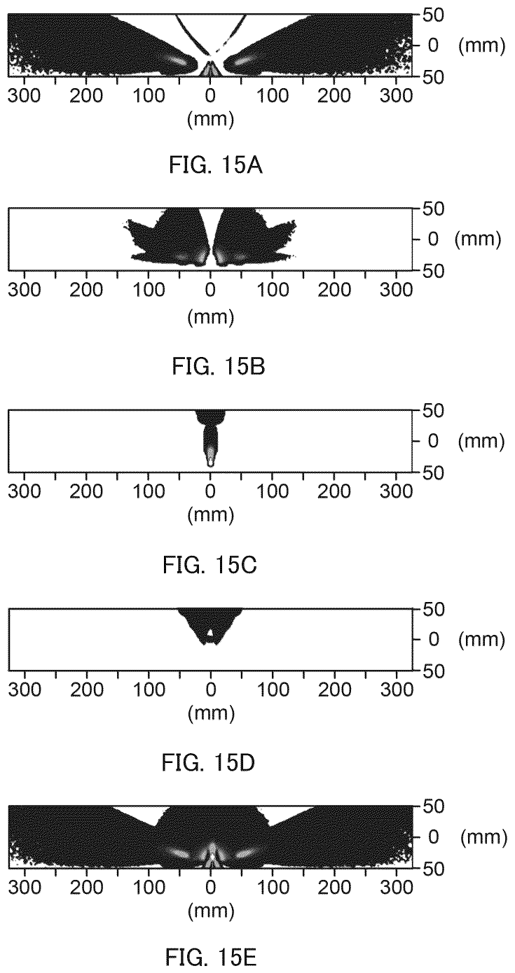

FIGS. 15A to 15E are diagrams illustrating an illuminance distribution of light at irradiated surface 120. FIG. 15A illustrates an illuminance distribution of light that is entered from first incidence surface 217 and second incidence surface 118 and is emitted from first emission surface 115, FIG. 15B illustrates an illuminance distribution of light that is entered from third incidence surface 119 and is emitted from first emission surface 115, FIG. 15C illustrates an illuminance distribution of light that is entered from first incidence surface 217 and second incidence surface 118 and is emitted from second emission surface 216, FIG. 15D illustrates an illuminance distribution of light that is entered from third incidence surface 119 and is emitted from second emission surface 216, and FIG. 15E illustrates an illuminance distribution of light that is entered from first incidence surface 217 and second incidence surface 118 and is emitted from first emission surface 115 and second emission surface 216. In FIGS. 15A to 15E, the abscissa indicates the distance from the center of major axis L1, and the ordinate indicates the distance from the center of minor axis L2.

As illustrated in FIGS. 14A and 15A, in a cross-section taken along a plane (XZ plane) including first axis A1 (X axis) and third axis A3 (Z axis), light emitted from the light emission center of light emitting element 111 with a small angle (emission angle) with respect to the optical axis of light emitting element 111 enters light flux controlling member 112 from first incidence surface 217. The light entered from first incidence surface 217 with a small angle with respect to optical axis OA reaches total reflection surface 114. The light having reached total reflection surface 114 is internally reflected toward first emission surface 115. The light reflected by total reflection surface 114 is emitted to the outside from first emission surface 115. In addition, as illustrated in FIG. 14A, the light entered from first incidence surface 217 with a large angle with respect to optical axis OA is emitted to the outside from first emission surface 115 without reaching total reflection surface 114.

As illustrated in FIGS. 14A and 15A, in a cross-section taken along the XZ plane, light emitted from the light emission center of light emitting element 111 with a further larger angle (emission angle) with respect to the optical axis of light emitting element 111 enters light flux controlling member 112 from second incidence surface 118. The light entered from second incidence surface 118 is emitted from first emission surface 115. At this time, in a cross-section taken along the XZ plane, the light that is emitted on the rear side (rear surface 122 side) than the vertex of second emission surface 216 is refracted toward the upper side. On the other hand, in a cross-section taken along the XZ plane, the light emitted on the front side than the vertex of second emission surface 216 advances with almost no refraction.

As illustrated in FIGS. 14B and 15D, in a cross-section taken along the YZ plane, a part of the light that is emitted from the light emission center of light emitting element 111 enters light flux controlling member 112 from third incidence surface 119. The light entered from third incidence surface 119 is emitted in a spreading manner at second emission surface 216. Note that in FIG. 14B, in a cross-section taken along the YZ plane, light emitted from the light emission center of light emitting element 111 and is emitted toward the left half with respect to optical axis OA is totally reflected by total reflection surface 114, and is therefore not illustrated.

In addition, as illustrated in FIG. 15B, a part of the light that is entered from third incidence surface 119 is emitted from first emission surface 115. In addition, as illustrated in FIG. 15C, a part of the light entered from first incidence surface 217 and second incidence surface 218 is emitted from second emission surface 216.

As illustrated in FIG. 15E, light that is entered from first incidence surface 217 and second incidence surface 218 and is emitted from first emission surface 115 and second emission surface 216 can irradiate the entirety of irradiated surface 120.

### Effects

As described above, irradiation device 100 of the present embodiment has the same effects as those of Embodiment 1.

### INDUSTRIAL APPLICABILITY

The light flux controlling member according to the present invention is applicable to the backlight of liquid crystal display devices, signs, generally-used illumination devices, and sterilization devices, for example.

### REFERENCE SIGNS LIST

100 Irradiation device
111 Light emitting element
112, 212 Light flux controlling member
113, 213 Incidence surface
114, 214 Total reflection surface
115 First emission surface
116, 216 Second emission surface
117, 217 First incidence surface
118, 218 Second incidence surface
119 Third incidence surface
120 Irradiated surface
121 Recess
122 Rear surface

The invention claimed is:

1. A light flux controlling member configured to control light emitted from a light emitting element so as to spread the light when the light flux controlling member is disposed over the light emitting element so as to intersect an optical axis of the light emitting element, the light flux controlling member comprising:
an incidence surface that is an inner surface of a recess that is open on a rear side, the incidence surface being configured to allow incidence of the light emitted from the light emitting element;
two total reflection surfaces disposed on a front side, and configured to reflect, away from the optical axis in a different direction, a part of light entered from the incidence surface;
two first emission surfaces configured to emit light reflected by the total reflection surface, to outside toward two directions along a first axis perpendicular to the optical axis; and
a second emission surface disposed at a part on a second axis perpendicular to the optical axis and the first axis between the two total reflection surfaces, the second emission surface being configured to emit another part of the light entered from the incidence surface, to the outside while spreading the light,
wherein the two total reflection surfaces are each a part of a surface that is obtained through rotation with a virtual half straight line as a rotation axis,
wherein the two virtual half straight lines each intersect the first emission surface,
wherein in plan view of the light flux controlling member, one ends of the two virtual half straight lines are located at the same point on the second axis, and an angle between the two virtual half straight lines is smaller than 180°, and
wherein in plan view of the light flux controlling member, the angle between the two virtual half straight lines is formed on a side on which an area of the second emission surface is larger with respect to the first axis.

2. The light flux controlling member according to claim 1,
wherein the incidence surface includes:
a first incidence surface disposed along the first axis to face the light emitting element;
a second incidence surface that intersects the first axis; and
a third incidence surface connecting the first incidence surface and an opening edge of the recess, and disposed at a position corresponding to the second emission surface, on one side with respect to the optical axis in a direction along the second axis,
wherein the first incidence surface allows the light emitted from the light emitting element to enter toward the total reflection surface or the first emission surface,
wherein the second incidence surface allows the light emitted from the light emitting element to enter toward the first emission surface, and
wherein the third incidence surface allows the light emitted from the light emitting element to enter toward the second emission surface.

3. The light flux controlling member according to claim 1, wherein in plan view of the light flux controlling member, the two virtual half straight lines are line-symmetric with respect to the second axis as a symmetry axis.

4. A light emitting device comprising:
a light emitting element; and
the light flux controlling member according to claim 1,
wherein when, in an XYZ coordinate system with three axes orthogonal to each other, the light emitting element is disposed such that the optical axis of the light emitting element coincides with a Z axis, and that a center of a light-emitting surface of the light emitting element is located at an origin of the XYZ coordinate system, and the light flux controlling member is disposed such that the first axis coincides with an X axis, and that the side on which the area of the second emission surface area is larger with respect to the X axis is located on a plus side in a Y axis, the two virtual half straight lines each pass through the following coordinates (XA, YA, ZA) and (XB, YB, ZB) that satisfy Condition (1) or Condition (2):
XA>0,YA>0,ZA>0, and XB<0,YB>0,ZB>0, and (Condition 1)
XA>0,YA>0,ZA<0, and XB<0,YB>0,ZB<0. (Condition2)

5. An irradiation device comprising:
a light emitting device including a light emitting element and a light flux controlling member configured to spread light emitted from the light emitting element; and
an irradiated surface including a major axis and a minor axis, and configured to be irradiated with the light emitted from the light emitting device,
wherein when, in an XYZ coordinate system with three axes orthogonal to each other, the light emitting element is disposed such that the optical axis of the light emitting element coincides with a Z axis, and that a center of a light-emitting surface of the light emitting element is located at an origin of the XYZ coordinate system, and the irradiated surface is disposed in an orientation of receiving the light emitted from the light emitting device so as to intersect the Z axis on a plus side in the Z axis with an X axis and the major axis being parallel to each other,
the light flux controlling member is symmetric about a YZ cross-section including a Y axis and the Z axis, and
the irradiation device has a luminance distribution with peaks in three directions toward points (Xn, Yn, Zn; n=1, 2 or 3) that satisfy the following conditions:
X1>0,Y1>0,Z1>0 and X1>Y1,X1>Z1 direction (Direction 1)
X2<0,Y2>0,Z2>0 and |X2|>Y2,|X2|>Z2 direction, and (Direction 2)
X3=0,Y3>0 and Z3>0 direction. (Direction 3)

6. The irradiation device according to claim 5, wherein a shortest distance between the light emitting element and the irradiated surface is shorter than the minor axis.

7. The irradiation device according to claim 5, wherein a center position in the minor axis of the irradiated surface is disposed on a plus side in the Y axis.

8. The irradiation device according to claim 5,
wherein a ratio of a length of the major axis and a length of the minor axis is 4:1 to 8:1, and
wherein in an XZ cross-section including the X axis and the Z axis, an angle between the optical axis and a straight line connecting the origin and an end portion of the irradiated surface is equal to or greater than 80° and smaller than 90°.

9. The irradiation device according to claim 5,
wherein the light flux controlling member includes:
an incidence surface that is an inner surface of a recess that is open on a rear side, the incidence surface being configured to allow incidence of the light emitted from the light emitting element;
two total reflection surfaces disposed on a front side, and configured to reflect, away from the optical axis in a different direction, a part of light entered from the incidence surface;
two first emission surfaces configured to emit light reflected by the total reflection surface, to outside toward two directions along a first axis perpendicular to the optical axis; and
a second emission surface disposed at a part on a second axis perpendicular to the optical axis and the first axis between the two total reflection surfaces, the second emission surface being configured to emit another part of the light entered from the incidence surface, to the outside while spreading the light,
wherein the two total reflection surfaces are each a part of a surface that is obtained through rotation with a virtual half straight line as a rotation axis,
wherein the two virtual half straight lines each intersect the first emission surface,
wherein in plan view of the light flux controlling member, one ends of the two virtual half straight lines are located at the same point on the second axis, and an angle between the two virtual half straight lines is smaller than 180°, and
wherein in plan view of the light flux controlling member, the angle between the two virtual half straight lines is formed on a side on which an area of the second emission surface is larger with respect to the first axis.

10. The irradiation device according to claim 5, wherein the light emitted from the light emitting element is an ultraviolet ray.

11. A sterilization device comprising the irradiation device according to claim 10.
